(19) United States
(12) Patent Application Publication
(10) Pub. No.: US 2013/0178495 A1
(43) Pub. Date: Jul. 11, 2013

(54) IBUDILAST FOR RELIEVING NEGATIVE EFFECTS OF MEDICATION OVERUSE HEADACHE

(71) Applicants: Adelaide Research & Innovation Pty Ltd., Adelaide (AU); MediciNova, Inc., San Diego, CA (US)

(72) Inventors: Paul ROLAN, Adelaide (AU); Kirk W. Johnson, Moraga, CA (US)

(73) Assignees: MediciNova, Inc., San Diego, CA (US); Adelaide Research & Innovation Pty Ltd., Adelaide (AU)

(21) Appl. No.: 13/712,818

(22) Filed: Dec. 12, 2012

Related U.S. Application Data
(60) Provisional application No. 61/576,183, filed on Dec. 15, 2011.

(30) Foreign Application Priority Data
Mar. 16, 2012 (AU) 2012201588
Mar. 16, 2012 (CA) 2,771,798

(51) Int. Cl. A61K 31/437

(52) U.S. Cl. CPC A61K 31/437
USPC 514/300

(57) ABSTRACT

Certain negative effects of medication overuse headache (MOH) are relieved by administering ibudilast or a pharmaceutically acceptable salt thereof.

### CROSS-REFERENCE TO RELATED PATENT APPLICATIONS

This application claims the benefit of the priority date of Provisional U.S. Application No. 61/576,183, filed Dec. 15, 2011, Australian Application No. 2012/201588, filed Mar. 16, 2012, and Canadian Application No. 2,771,798, filed Mar. 16, 2012, the contents of each of which are incorporated by reference herein in their entirety.

### FIELD OF THE INVENTION

This invention relates to relieving certain negative effects of medication overuse headache (MOH) by administering ibudilast or a pharmaceutically acceptable salt thereof.

### BACKGROUND OF THE INVENTION

Medication overuse headache (MOH) is a chronic disorder, which significantly impacts the quality of life of affected individuals, and imposes a large economic burden upon the society. Medication overuse headache is reported to be the third most common form of headache, following only tension type headache (TTH) and migraine. Globally, between 1-2% of the general adult population suffer chronic headache associated with medication overuse. However, the prevalence of MOH in specialist headache centers is much higher. Patients with chronic daily headache, including medication overuse headache, typically score between 10-25% lower than episodic headache sufferers in measures of vitality, work capacity, mental health and body pain. Ibudilast is an agent that has been reported, or contemplated, to be used in pain models and patients suffering from pain.

Shimomura et al., Headache, 31 (7): 483, 1991, described a female patient suffering from chronic-tension type headaches every day. For over six months, she had taken Sedes G®, one to two grams per day, containing per gram of Sedes G®, 150 mg of isopropylantipyrin, 60 mg of allylisopropylacetylurea, 250 mg of phenacetin, and 50 mg of caffeine. Reportedly, she complained of different types of headaches, bilateral, occipital, and pulsatile, lasting for over 48 hours. Her headaches were severe when she forgot to take Sedes G®. The Shimomura et al. reference reports explaining to the patient that her headaches were induced by Sedes G®. The patient was treated with 20 mg per day of ibudilast. Reportedly, her daily headaches disappeared in one week. She stopped taking Sedes G®. Her migraine on the right side continued, but the frequency and the intensity of her migraine decreased.

The reference Bartley, Medical Hypotheses, 72, 255-257, 2009, hypothesized that the patient in the Shimomura et al. reference was a migraine sufferer with MOH, who was treated with ibudilast. However, even this hypothesis is based on the treatment outcome of a single patient performed without a control patient. The Shimomura et al. reference does not report if the patient's headache developed or worsened during Sedes G® use, and was silent about opioid overuse related MOH in humans and relieving its negative effects. Given the amount of caffeine that this patient consumed daily, relatedness to caffeine use or withdrawal is a possibility.

U.S. patent application no. 2008/0181876 described a method for enhancing the analgesic efficacy of an opioid in a subject, comprising administering to the subject an effective amount of a phosphodiesterase inhibitor, or a glial attenuator that potentiates opioid-induced analgesia in the subject. It also described ibudilast as the phosphodiesterase inhibitor or the glial attenuator. U.S. Pat. No. 7,534,806 described a method for treating neuropathic pain with ibudilast, and also described administering morphine and related natural opioids (opiates) for this purpose. However, the '876 application and the '806 patent are silent about treating negative effects and manifestation of MOH. As such, every type of pain that requires analgesia, or every type of neuropathic pain does necessarily not lead to or arise from MOH.

A reference, Wieseler et al., IASP, The 13th World Congress on Pain, 2010, (Wieseler 1) described testing minocycline and ibudilast in a rat model of facial allodynia that placed bilateral indwelling catheters between the meninges and the skull (Wieseler et al., J. Neurosci. Methods, 2009 (Wieseler 2)). The Wieseler 1 reference reported that minocycline and ibudilast blocked and reversed facial allodynia in the rat model, and concluded that the data reported in the reference supported a modulatory role for glia in facial allodynia induced by supradural inflammatory mediators. However, the references did not suggest any link between the animal model and opioid overuse related MOH in humans; neither did the references suggest any link between blocking and reversing facial allodynia with relieving opioid overuse related negative effects of MOH.

The ClinicalTrials.gov website reported a prospective clinical trial for testing ibudilast for migraine headache. http ://clinicaltrials.gov/ct2/show/NCT01389193. However, there was no mention of opioid overuse related MOH in humans and relieving its negative effects.

The present invention arises in part out of the discovery that ibudilast is useful for relieving one or more negative effects of MOH.

### SUMMARY OF THE INVENTION

In one aspect, the present invention provides a method of relieving one or more negative effects of medication overuse headache (MOH) in a patient suffering from a headache and using an analgesic agent comprising a natural or synthetic opioid in a way that contributes to or gives rise to the headache, the method comprising administering to a patient in need thereof an effective amount of ibudilast or a pharmaceutically acceptable salt thereof.

In another aspect, the present invention provides a method of relieving one or more negative effects of opioid overuse-related medication overuse headache (MOH) in a patient having a history of opioid overuse and experiencing chronic daily headache, the method comprising administering to a patient in need thereof an effective amount of ibudilast or a pharmaceutically acceptable salt thereof.

In another aspect, the present invention provides a method of reducing opioid use by a patient having a history of opioid overuse and exhibiting one or more negative effects, including chronic daily headache, the method comprising administering to a patient in need thereof an effective amount of ibudilast or a pharmaceutically acceptable salt thereof.

In another aspect, the present invention provides a method of inhibiting the formation of an opioid dependence in a patient having a history of overuse of an analgesic agent comprising a natural or synthetic opioid and exhibiting one or more negative effects, including chronic daily headache, the method comprising administering to a patient in need thereof an effective amount of ibudilast or a pharmaceutically acceptable salt thereof.

In another aspect, the present invention provides a method of improving cognition and productivity in a patient having a history of overuse of an analgesic agent comprising a natural or synthetic opioid and exhibiting one or more negative effects, including chronic daily headache, the method comprising administering to a patient in need thereof an effective amount of ibudilast or a pharmaceutically acceptable salt thereof.

In another aspect, the present invention provides a method of weaning a patient off of an analgesic agent comprising a natural or synthetic opioid, the patient having a history of overuse of the analgesic agent and exhibiting one or more negative effects, including chronic daily headache, the method comprising administering to a patient in need thereof an effective amount of ibudilast or a pharmaceutically acceptable salt thereof.

### BRIEF DESCRIPTION OF THE DRAWING

FIG. 1 illustrates a flow chart of testing ibudilast according to the present invention as exemplified in an example below.

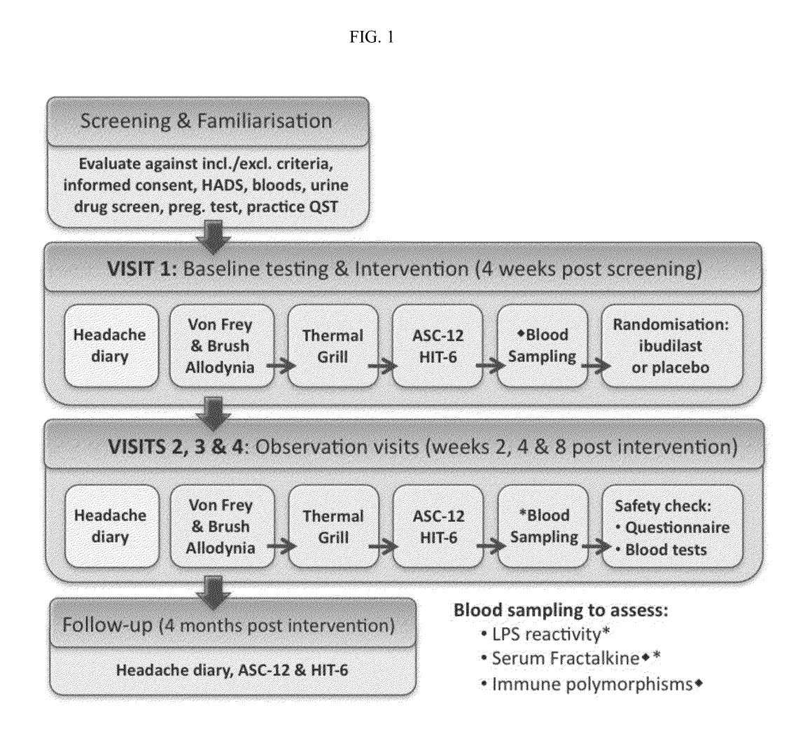

### DETAILED DESCRIPTION OF THE INVENTION

### Definitions

“Administering” or “administration of an agent to a patient (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

“Chronic daily headache” refers to a headache occurring for at least 15 days per month for at least 3 months, preferably in a row, and lasting at least 4 hours if untreated. Chronic daily headache, with the added criterion of medical overuse, can lead to medical overuse headache.

“Effective amount” of an agent refers to an amount of the agent that, when administered to a patient with MOH, will have the intended therapeutic effect, e.g., alleviation, amelioration, palliation, reduction, or elimination of one or more negative effects of MOH in the patient. The full therapeutic effect does not necessarily occur by administration of one dose (or dosage), and may occur only after administration of a series of doses. Thus, an effective amount may be administered in one or more administrations.

“Ibudilast” refers to a compound of formula:.

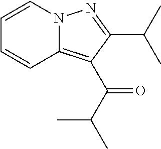 [SMILES: CC(C)C(=O)c1c(C(C)C)nn2ccccc12]

“Medication overuse headache” (MOH) refers to a condition diagnosed in patients with A) headache on ≧15 days per month, B) regular overuse of acute headache treatments for >3 months, and C) for whom headache has developed or has worsened during medication overuse. Medication intake on ≧10 days per month is considered overuse for ergotamine, triptans, opioids and combination preparations, whereas intake on ≧15 days per month is considered to meet the criteria for overuse of simple analgesics (non-steroidal anti-inflammatory drugs (NSAIDs), aspirin and paracetamol) or a combination of acute headache treatments.

“Opioid” refers to a compound that works pharmacologically by binding to opioid receptors. Non limiting examples of opioids include, morphine, codeine, thebaine, heroin, hydromorphone, hydrocodone, oxycodone, oxymorphone, desomorphine, nicomorphine, dipropanoylmorphine, benzylmorphine, ethylmorphine, buprenorphine, fentanyl, pethidine, methadone, tramadol, and dextropropoxyphene. Natural opioids or opiates are opioids found in the opium poppy plant. Synthetic opioids are compounds that are not obtained from natural sources, such as fentanyl, or are synthetically modified derivatives, such as hydrocodone, of opiates, such as codeine and thebaine.

“Patient” refers to any mammal, such as rodents, simians, dogs, and humans.

“Pharmaceutically acceptable salts” refer to any salt of ibudilast that is non-toxic and safe for in vivo administration to a patient, including a human patient.

“Reduction” of one or more negative effects (and grammatical equivalents of this phrase) refers to decreasing the severity and/or frequency of the negative effect(s), and/or elimination of the negative effects(s).

### Preferred Embodiments

In one aspect, the present invention provides a method of relieving one or more negative effects of medication overuse headache (MOH) in a patient suffering from a headache and using an analgesic agent comprising a natural or synthetic opioid in a way that contributes to or gives rise to the headache, the method comprising administering to a patient in need thereof an effective amount of ibudilast or a pharmaceutically acceptable salt thereof.

In another aspect, the present invention provides a method of relieving one or more negative effects of opioid overuse-related medication overuse headache (MOH) in the patient having a history of opioid overuse and experiencing chronic daily headache, the method comprising administering to a patient in need thereof an effective amount of ibudilast or a pharmaceutically acceptable salt thereof.

As used herein, relieving refers to one or more of alleviation, amelioration, palliation, reduction, or elimination of one or more negative effects of MOH in the patient

In another aspect, the present invention provides a method of inhibiting the onset of one or more negative effects of medication overuse headache (MOH) in a patient suffering from bouts of headache and using an analgesic agent comprising a natural or synthetic opioid in a way that contributes to or gives rise to bouts of headache, the method comprising administering to a patient in need thereof MOH an effective amount of ibudilast or a pharmaceutically acceptable salt thereof.

In another aspect, the present invention provides a method of reducing opioid use by a patient having a history of opioid overuse and exhibiting one or more negative effects, including chronic daily headache, the method comprising administering to a patient in need thereof an effective amount of ibudilast or a pharmaceutically acceptable salt thereof.

In another aspect, the present invention provides a method of inhibiting the formation of an opioid dependence in a patient having a history of overuse of an analgesic agent comprising a natural or synthetic opioid and exhibiting one or more negative effects, including chronic daily headache, the method comprising administering to a patient in need thereof an effective amount of ibudilast or a pharmaceutically acceptable salt thereof.

In another aspect, the present invention provides a method of improving cognition and productivity in a patient having a history of overuse of an analgesic agent comprising a natural or synthetic opioid and exhibiting one or more negative effects, including chronic daily headache, the method comprising administering to a patient in need thereof an effective amount of ibudilast or a pharmaceutically acceptable salt thereof.

As used herein, “history of overuse” refers to using the medication for at least 3 months, preferably, for 6 months or more.

In another aspect, the present invention provides a method of weaning a patient off of an analgesic agent comprising a natural or synthetic opioid, the patient having a history of overuse of the analgesic agent and exhibiting one or more negative effects, including chronic daily headache, the method comprising administering to a patient in need thereof an effective amount of ibudilast or a pharmaceutically acceptable salt thereof.

In one embodiment, the patient uses the analgesic agent at least 10 days/month for at least the last three months. In one embodiment, the patient uses the analgesic agent at least 15 days/month for at least the last three months. In one embodiment, the patient uses the analgesic agent at least 25 days/month for at least the last three months. In another embodiment, the patient suffers a headache at least 15 days/month for at least the last two months. In another embodiment, the patient suffers a headache at least 25 days/month for at least the last two months. In another embodiment, the intensity and/or the frequency of the patient's headache developed or worsened with use of the analgesic agent.

In another embodiment, the patient is not concurrently suffering from neuropathic pain, arthritic pain, cancer-caused pain, or coughs. In yet another embodiment of the invention, the patient is not concurrently suffering from heavy caffeine use or withdrawal.

In another embodiment, the natural or synthetic opioid is selected from the group consisting of morphine, codeine, thebaine, heroin, hydromorphone, hydrocodone, oxycodone, oxymorphone, desomorphine, nicomorphine, dipropanoylmorphine, benzylmorphine, ethylmorphine, buprenorphine, fentanyl, pethidine, methadone, tramadol, dextropropoxyphene and mixtures of one or more of the foregoing. In another embodiment, the analgesic agent comprises codeine. In another embodiment, the analgesic agent is codeine. In another embodiment, the analgesic agent does not include a non-steroidal anti-inflammatory agent

In another embodiment, the one or more negative efffects of MOH that are relieved according to the present invention include allodynia, depression, anxiety, neck pain, central sensitivity, acute medication administration frequency, headache frequency, duration of headache, intensity of headache, or frequency of migraine attacks.

In another embodiment, the allodynia includes thermal allodynia or thermal hyperalgesia. In another embodiment, the thermal allodynia or hyperalgesia results from a heat or a cold stimulus. In another embodiment, the allodynia includes mechanical allodynia. In another embodiment, the allodynia includes cutaneous allodynia. In another embodiment, the cutaneous allodynia includes cephalic cutaneous allodynia. In another embodiment, the cutaneous allodynia includes extra-cephalic cutaneous allodynia.

In another embodiment, the ibudilast or the pharmaceutically acceptable salt thereof is administered in an amount ranging from about 40-120 mg of free base ibudilast equivalent/day, preferably about 40-100 mg of free base ibudilast equivalent/day, more preferably about 60-100 mg of free base ibudilast equivalent/day, and yet more preferably about 80 mg of free base ibudilast equivalent/day. In another embodiment, the ibudilast or the pharmaceutically acceptable salt thereof is administered once daily, twice daily, thrice daily, or once every 2-3 days. In another embodiment, the ibudilast or the pharmaceutically acceptable salt thereof is administered daily for up to 4 weeks, up to 8 weeks, up to 16 weeks, or up to 32 weeks. In another embodiment, the ibudilast or the pharmaceutically acceptable salt thereof is administered enterally, preferably orally, or topically, preferably transdermally.

### EXAMPLE

This example illustrates the use of ibudilast according to the present invention for relieving negative effects of MOH in patients.

### Test Overview

A double-blind, randomised, placebo-controlled, pilot test of ibudilast for relieving one or more negative effects of medication overuse headache is contemplated. The test will involve a screening visit followed by four visits to the test center for baseline testing, initiation of the test drug administration, and ongoing data collection during and at the completion of treatment. Follow up data will be collected by correspondence 4 months after treatment is completed, following a reminder at 3 months.

During the baseline visit, quantitative sensory testing (QST) will be carried out using three different experimental pain models to obtain a clear picture of each patient's pain sensitivity. Comprehensive medication and headache histories will be taken, and evaluation of plasma fractalkine levels, ex vivo response of peripheral blood mononuclear cells (PBMCs) to lipopolysaccharide (LPS) and immune system polymorphisms will take place. A blood sample will also be obtained from patients to assess CYP2D6 genotype and associated metabolic status.

Following baseline assessments, patients are randomized (in a 1:1 ratio) to commence either ibudilast or placebo treatment, which will continue for eight weeks. During, and at the completion of ibudilast/placebo treatment, (weeks 2, 4 and 8), patients will return to the test center at which time QST and the blood assays conducted at baseline (excluding genotyping) will be repeated. Patients will complete a headache diary for 2 weeks prior to baseline assessments, during the treatment period, and for 4 weeks prior to the follow up data collection at 4 months post treatment. The diary will record headache frequency, duration, intensity, pain characteristics, and medication intake for comparison with baseline data.

### Screening Visit

Prior to test enrolment, the suitability of each patient will be assessed according to certain inclusion and exclusion criteria.

### Patient Inclusion Criteria

Patients will be eligible for study enrolment if all of the following criteria apply:
Regular use, for at least 3 months, of codeine-containing analgesics on ≧10 days/month
Headache present on at least 15 days/month, for at least 2 months
Headache developed or markedly worsened during medication overuse
Primary indication for analgesics is headache disorder

### Patient Exclusion Criteria

Patients will be ineligible for study enrolment if any of the following criteria apply:
Receiving tramadol or opioid medications other than codeine
Taking triptans >4 days/month
Taking codeine for reasons other than headache (e.g. for treating other pain conditions, cough, or bowel motility)
Severe psychiatric disorders
Other chronic pain conditions (e.g. neuropathic pain, arthritis)
Diabetic neuropathy
Recent or current active infection, determined to be clinically significant by the principal investigator
Known active inflammatory diseases such as rheumatoid arthritis
History of cerebrovascular disorder
Recent history of significant trauma, as determined by the principal investigator including major surgery within the previous 2 months
Recent history of drug or alcohol abuse
Spinal cord injury
Any clinically significant findings on screening blood sample results
Current malignancy
Known hypersensitivity to ibudilast or excipients in its formulation
Renal or hepatic impairment, defined as baseline GFR (as calculated by the Cockcroft-Gault equation) of <60 mL/min, LFTs (excluding bilirubin) >3 times the upper limit of normal or bilirubin >2 times the upper limit of normal
For females of childbearing potential:
   Pregnancy
   Lack of adequate contraception (abstinence, double barrier method, intrauterine device, surgical sterilization (self or partner), hormonal contraceptive methods (oral, injected, or implanted)
   Breastfeeding

Screening will consist of:
Medical history
Headache diagnosis based upon retrospective information from patient (to be confirmed with headache diary data at baseline visit)
Extensive medication history
Patient completion of the Hospital Anxiety and Depression Scale (HADS)
Physical examination
Urine pregnancy test for women of childbearing potential
Urine drug screen to confirm presence of codeine/metabolites and other non-prescribed drugs of abuse
Blood samples to assess haematology (4 mL), biochemistry (4 mL) including renal function (serum creatinine (Cr_{Se})), and hepatic function (liver function tests (LFTs)) (5 mL)

Once enrolled in the test, an additional blood sample (30 mL) will be obtained to assess CYP2D6 genotype and associated metabolic status and to determine the presence of polymorphisms in the immune system likely to result in increased activity. Patients will then be provided with a headache diary to complete during the 4 weeks prior to the baseline visit. Patients will be asked to fill in the headache diary once a day. Standardized education and instructions on how to complete the diary will be presented to all patients. The headache diary will record, head pain characteristics, headache frequency, average headache intensity (11-point numerical rating scale (NRS)), duration of headache (number of hours) and intake of symptomatic headache treatments (timing, type, amount of medication consumed). The screening will take place between 28 and 35 days prior to the scheduled baseline assessment.

### Familiarization Session

Following screening, eligible patients will remain at the test centre to complete a brief familiarisation session, to ensure that they are accustomed to the experimental procedures of the quantitative sensory testing (von Frey filament test, brush allodynia test, thermal grill illusion).

### Baseline Visit

During the first visit, patients' pain sensitivity will be evaluated at cephalic and extra-cephalic sites using three different methods of quantitative sensory testing. The von Frey filament test and the brush allodynia test will be employed to assess sensitivity to both static and dynamic mechanical pain respectively. Both the von Frey test and the brush allodynia test will be performed at the same anatomical sites. The test sites will include the left and rights sides of the forehead, each cheek, the left and right sides of the jaw and both inner forearms, assessed in a randomized order.

The thermal grill illusion will also be utilized to determine central sensitivity to pain, without activating peripheral nociceptors, on both sides of the face and on each hand. Prior to assessment with the thermal grill, each patient's cold and heat pain thresholds will be obtained using a thermode to ensure the temperatures tested are non-noxious. The temperature combinations of 22° C. and 38° C. as well as 18° C. and 42° C. will be investigated.

Participants will be asked to place their left cheek, right cheek, left palm or right palm on the thermal grill, orthogonally to the long axis of the bars with moderate pressure for 30 seconds. Therefore, each participant will perform 8 assessments with the thermal grill in a randomized order, to avoid any possible period effect. Furthermore, additional randomisation will be performed to ensure that the same limb is not assessed in consecutive order.

Prior to thermal grill testing, participants will be required to rate the pain they experience on average from their headache. Immediately after contact with the thermal grill, participants will be required to rate the intensity of pain produced by the thermal grill on an 11-point NRS and a 100 mm visual analogue scale (VAS); intensity of heat on a 11-point NRS and a 100 mm VAS colour bar; unpleasantness produced by the thermal grill on an 11-point NRS; their tolerability to the thermal grill on a 100 mm VAS; characterise the sensation experienced by the thermal grill; and rate ‘how close the bars felt to burning you’ and ‘how similar was the intensity of pain experienced from the temperature bars to the intensity of pain you experience from your headache’. Participants will also be given an opportunity to write about the sensation(s) they experienced.

All QST will be carried out in accordance with well known standard operating procedures. Patients will then complete two brief self-administered questionnaires. Patients will complete the 6-point Headache Impact Test (HIT-6) to evaluate the impact of headache on quality of life, reflecting upon the previous month and the 12-point Allodynia Symptom Checklist (ASC-12) to indicate if, and quantify how, increased pain sensitivity impacts upon activities of day-to-day life.

Blood samples will be taken to perform a number of tests including:
Evaluation of ex vivo response of PBMCs to LPS (18 mL)
Measurement of plasma fractalkine (9 mL)
Haematology, biochemistry, and renal and hepatic function (13 mL)
   A further 20 mL blood sample will also be taken, de-identified and stored for possible pain biomarker analysis at a later date.

During this visit, headache diary entries for the 4 week pre-test period will be collected to confirm test eligibility, and retained for comparison with future entries. Patients who are eligible to continue in the test will then be randomized to either ibudilast or placebo treatment groups. Test medication will be dispensed to the patients. At the completion of this session, patients will be given standardized education, they will be advised it is best to take only the minimum amount of medication required to control their pain, however they will not specifically be instructed to reduce their analgesic intake. Patients will again be asked to complete the provided headache diary for the 8-week treatment period.

### Observation Visits

At weeks 2 and 4, and at the completion of ibudilast/placebo treatment (week 8), QST and blood testing will be repeated following identical procedures to those used during the baseline test visit. Patients will be asked to bring their diary and test medication to each visit for review and accountability purposes. At week 8 the headache diary and any unused medication will be returned. Patients will again complete the HIT-6 and ASC-12 at weeks 4 and 8.

During the observational visits patients will be asked to report any adverse effects and blood biochemistry (including assessment of renal function), LFTs and haematology will be monitored as an additional safety measure.

### Follow Up Data Collection

At 6 months from the initiation of ibudilast/placebo treatment, follow up data will be collected. Three months after completion of treatment, patients will be contacted by telephone and asked to again complete a 4-week headache diary, which will be posted to them. Included with the headache diary will be the HIT-6 and the ASC-12 questionnaires to be completed completion 4 months after the treatment period has ended.

### Test Drug and its Administration

Ibudilast, as a 10 mg unit delayed-release dose form will be used in this testing. This could include commercial formulations such as Pinatos® or Ketas® capsules. An ibudilast dose of 40 or 50 mg twice daily, titrated from 30 mg twice daily over 3-5 days, is contemplated for use in this test. The placebo capsules will contain microcrystalline cellulose. The test drug ibudilast/placebo will be self administered by patients orally, twice daily. Patients will be given a supply of capsules to last the entire 8 weeks of treatment.

### Primary Efficacy End Point

With preferred efficacy goals that include decreasing the frequency, severity and duration of headaches, the primary end point for this test will be change from baseline in headache index, as calculated by the summation of headache duration (hours) X headache intensity (11-point numerical rating scale).

### Secondary Efficacy End Points

The secondary end points assessed will include:
Medication frequency (number of days acute headache medication taken/month)
Headache frequency (number of days with headache/month)
Duration of headache (hours)
Intensity of headache (numerical rating scale)
Frequency of probable migraine attacks (number of attacks/month)
Headache related impact on quality of life as assessed using the HIT-6, which is based upon Kosinski, M. et al., (2003) “A six-item short-form survey for measuring headache impact: The HIT-6™” Quality of Life Research, 12, 963-974, incorporated herein by reference.
Cutaneous allodynia as assessed using the ASC-12 (see, Lipton, R. et al, (2008) “Cutaneous allodynia in the migraine population.” Annals of Neurology, 63, 148-158, incorporated herein by reference) and QST
Response rate (response defined as ≧30% reduction in headache days/month or headache index from baseline)
   Percentage of patients who see a >30% reduction in headache index after ibudilast treatment (at week 8)
   NNT, number of patients treated to see 1 patient “respond” as above
Relapse rate, expressed as the percentage of patients who were classed as responders at 1 month who no longer meet the criteria for response at 6 months.

1. A method of relieving one or more negative effects of medication overuse headache (MOH) in a patient suffering from a headache and using an analgesic agent comprising a natural or synthetic opioid in a way that contributes to or gives rise to the headache, the method comprising administering to a patient in need thereof an effective amount of ibudilast or a pharmaceutically acceptable salt thereof.

2. The method of claim 1 in which the natural or synthetic opioid is selected from the group consisting of codeine, morphine, thebaine, heroin, hydromorphone, hydrocodone, oxycodone, oxymorphone, desomorphine, nicomorphine, dipropanoylmorphine, benzylmorphine, ethylmorphine, buprenorphine, fentanyl, pethidine, methadone, tramadol, dextropropoxyphene and mixtures of one or more of the foregoing.

3. A method of inhibiting the onset of one or more negative effects of medication overuse headache (MOH) in a patient suffering from bouts of headache and using an analgesic agent comprising a natural or synthetic opioid in a way that contributes to or gives rise to bouts of headache, the method comprising administering to a patient in need thereof an effective amount of ibudilast or a pharmaceutically acceptable salt thereof.

4. The method of claim 3 in which the natural or synthetic opioid is selected from the group consisting of codeine, morphine, thebaine, heroin, hydromorphone, hydrocodone, oxycodone, oxymorphone, desomorphine, nicomorphine, dipropanoylmorphine, benzylmorphine, ethylmorphine, buprenorphine, fentanyl, pethidine, methadone, tramadol, dextropropoxyphene and mixtures of one or more of the foregoing.

5. A method of relieving one or more negative effects of opioid overuse-related medication overuse headache (MOH) in a patient having a history of opioid overuse and experiencing chronic daily headache, the method comprising administering to a patient in need thereof an effective amount of ibudilast or a pharmaceutically acceptable salt thereof.

6. A method of reducing opioid use by a patient having a history of opioid overuse and exhibiting one or more negative effects, including chronic daily headache, the method comprising administering to a patient in need thereof an effective amount of ibudilast or a pharmaceutically acceptable salt thereof.

7. The method of claim 1 in which the patient uses the analgesic agent at least 10 days/month for at least the last three months.

8. The method of claim 1 in which the patient suffers a headache at least 15 days/month for at least the last two months.

9. The method of claim 1 in which the patient's headache developed or worsened with use of the analgesic agent.

10. The method of claim 1 in which the patient is not concurrently suffering from neuropathic pain, arthritic pain, cancer-caused pain, or coughs.

11. The method of claim 1 in which the analgesic agent comprises codeine.

12. The method of claim 1 in which the one or more negative effects of MOH relieved include allodynia, thermal hyperalgesia, depression, anxiety, neck pain, central sensitivity, acute medication administration frequency, headache frequency, duration of headache, intensity of headache, or frequency of migraine attacks.

13. The method of claim 12 in which the allodynia includes thermal allodynia or thermal hyperalgesia.

14. The method of claim 13 in which the thermal hyperalgesia results from a heat or a cold stimulus.

15. The method of claim 12 in which the allodynia includes mechanical allodynia.

16. The method of claim 15 in which the allodynia includes cutaneous allodynia.

17. The method of claim 16 in which the cutaneous allodynia includes cephalic cutaneous allodynia.

18. The method of claim 16 in which the cutaneous allodynia includes extra-cephalic cutaneous allodynia.

19. The method of claim 1 in which the ibudilast or a pharmaceutically acceptable salt thereof is administered in an amount ranging from about 40-120 mg of free base ibudilast equivalent/day.

20. The method of claim 1 in which the ibudilast or a pharmaceutically acceptable salt thereof is administered once daily, twice daily, thrice daily, or once every 2-3 days.

21. The method of claim 1 in which the ibudilast or a pharmaceutically acceptable salt thereof is administered daily for up to 4 weeks, up to 8 weeks, up to 16 weeks, or up to 32 weeks.

22. The method of claim 1 in which the ibudilast or a pharmaceutically acceptable salt thereof is administered enterally or topically.

23. A method of inhibiting the formation of an opioid dependence in a patient having a history of overuse of an analgesic agent comprising a natural or synthetic opioid and exhibiting one or more negative MOH effects, including chronic daily headache, the method comprising administering to a patient in need thereof an effective amount of ibudilast or a pharmaceutically acceptable salt thereof.

24. A method of improving cognition and productivity in a patient having a history of overuse of an analgesic agent comprising a natural or synthetic opioid and exhibiting one or more negative MOH effects, including chronic daily headache, the method comprising administering to a patient in need thereof an effective amount of ibudilast or a pharmaceutically acceptable salt thereof.

25. A method of weaning a patient off of an analgesic agent comprising a natural or synthetic opioid, the patient having a history of overuse of the analgesic agent and exhibiting one or more negative effects, including chronic daily headache, the method comprising administering to a patient in need thereof an effective amount of ibudilast or a pharmaceutically acceptable salt thereof.
